(19) United States
(12) Patent Application Publication
(10) Pub. No.: US 2021/0277025 A1
(43) Pub. Date: Sep. 9, 2021

(54) BIONANOCOMPOSITE AND METHOD THEREOF

(71) Applicant: NATIONAL INSTITUTE OF FOOD TECHNOLOGY ENTREPRENEURSHIP AND MANAGEMENT-NIFTEM, Sonipat (IN)

(72) Inventors: Divya Sachdev, Pitampura (IN); Akshay Bisht, Rohini Delhi (IN); Renu Pasricha, Dwarka (IN); Neetu Kumra Taneja, Sonipat (IN); J.S. Rana, Sonipat (IN)

(21) Appl. No.: 17/196,233

(22) Filed: Mar. 9, 2021

(30) Foreign Application Priority Data
Mar. 9, 2020 (IN) 202011010076

(51) Int. Cl. C07F 3/06; B09B 3/00; G01N 21/64; G01N 27/447

(52) U.S. Cl. CPC C07F 3/06; B09B 3/0016; B82Y 5/00; G01N 27/44726; G01N 21/6458

(57) ABSTRACT

A fluorescent bionanocomposite is provided, as well as a composition and method of synthesis of fluorescent bionanocomposite for selective stain for gram negative bacteria.

This application claims priority to Indian Patent Application No. 202011010076, filed on Mar. 9, 2020, the disclosure of which is incorporated herein by reference.

### FIELD OF INVENTION

The present invention relates to a fluorescent bionanocomposite. More specifically, the Invention provides a composition and method of synthesis of fluorescent bionanocomposite for selective stain for gram negative bacteria.

### BACKGROUND OF THE INVENTION

The following background discussion includes information that may be useful in understanding the present invention. It is not an admission that any of the information provided herein is prior art or relevant to the presently claimed invention, or that any publication specifically or implicitly referenced is prior art Fluorescent nanomaterial based selective stain possesses high specificity and sensitivity for Staining of bacterial cells. Traditionally, staining of bacterial cell is a routinely used auxiliary method for their classification, visualisation of cellular morphology, detection, identification, labeling and targeting. Bacterial Gram-staining is the gold standard technique comprising the use of multiple dyes/reagents providing specific and selective identification of bacteria (Gram positive/Gram negative) based up on differences in cells colour and morphology.

The requirement of time monitoring and tedious sequential steps makes the Gram's staining technique cumbersome, requiring technical expertise to achieve reliable results. Hence the demand is for faster, selective fluorescent stains. The increase of available fluorescent stains together with the use of a fluorescent microscope or high-end confocal scanning laser microscopy is used via interaction between both viable and fixed bacteria. Although fluorescent stains have been reported to possess high specificity and sensitivity, however most of the stains available in the market suffer from limitations of photo stability, quenching of signal during imaging, toxicity, restrictive usage of conducting the staining in dark condition and stability. Further the available fluorescent stain is not environment friendly.

Hence there is a need of such fluorescent probe which is synthesized from biological waste (agriculture) and uses greener method for synthesis and shows the non-toxic behavior and can be used for stain detection. There is also needed a method for the detection of bacteria which is efficient, avoids the cumbersome, time consuming procedures used conventionally that often compromises reliability and reproducibility and is based on an eco-friendly, bio-degradable fluorescent probe.

### SUMMARY

Primary object of the present invention is to overcome the limitation of available fluorescent stains available in the market.

Another object of the present invention is to provide a novel biological derived fluorescent nanoprobe for detecting Gram negative bacteria.

Yet another object of the present invention is to provide a fluorescent nanoprobe for various applications.

Yet another object of the present invention is to provide a nanoprobe which offers a rapid, sensitive and universal fluorescent detection of Gram negative bacteria in situ condition.

Yet another object of the present invention is to provide an eco friendly, bio degradable, fluorescent probe.

Yet another object of the present invention is to provide a fluorescent stain which can detect the gram negative bacteria within a short span of time.

Yet another object of the present invention is to provide optimum concentration of nano probe utilized to stain bacteria is very low.

Yet another object of the present invention is to provide method for the detection of bacteria which is efficient, avoids the cumbersome, time consuming procedures used conventionally that often compromises reliability and reproducibility and is based on an eco-friendly, bio-degradable fluorescent probe.

Yet another object of the present invention is to provide an environmentally safe method for staining nucleic acid.

It is to be understood that this invention is not limited to the particular systems, and methodologies described, as there can be multiple possible embodiments of the present invention which are not expressly illustrated in the present disclosure. It is also to be understood that the terminology used in the description is for the purpose of describing the particular versions or embodiments only, and is not intended to limit the scope of the present invention which will be limited only by the appended claims.

In an aspect, the invention provides a bio-degradable fluorescent zinc bionanocomposite (ZBN), synthesized from agriculture waste, for selective staining of gram negative bacteria comprising:
a) at least zinc salt present in an amount ranging from 2 mmol to 80 mmol;
b) extract of agriculture waste comprising an extract of straw, stems, peel and dried leaves of said agriculture waste in a solvent, present in an amount ranging from 15 ml to 50 ml where ratio of said extract of agriculture waste to zinc salt comprises 0.25:1 to 5:1.

In another aspect, the invention provides a method for the preparation of bio-degradable fluorescent zinc bionanocomposite (ZBN) as described above, comprising the steps of:
a) preparing a solution of zinc salt in said extract of agriculture waste in varied ratios followed by stirring to obtain a mixture, wherein the said mixture is heated at variable temperature for 45 min to 1 hour where said variable temperature comprises temperature ranging from 50° C. to 100° C.;
   where ratio of said extract of agriculture waste to zinc salt comprises 0.25:1 to 5:1;
b) adding the metal solution alkaline to the solution obtained in step (a) in the presence of an inert atmosphere where the formation of ZBN bionanocomposite containing nanoparticles is indicated by the colour change of the mixture from white to brown;
c) cooling the solution obtained in step (b) followed by filtering the washing and obtaining the nanoparticle of zinc bionanocomposite (ZBN).

In an aspect, the invention provides a method for the detection of gram negative bacteria by the bio-degradable fluorescent zinc bionanocomposite (ZBN) as described above, comprising:
a) treating the overnight grown bacterial culture with the suspension of 20 micro liter of said ZBN for approximately 5-10 minutes followed by washing said culture with water;
b) detecting the bacteria through fluorescence microscope.

In another aspect, the invention provides a method for the nucleic acid staining by the bio-degradable fluorescent ZBN as described above, comprising the steps of:

a) treating approximately 500 ng of nucleic acid sample with 5 μl of diluted ZBN solution for approximately 1-30 minutes;
b) adding 1× gel loading dye to the solution obtained from step (a) followed by running on agarose gel electrophoresis and visualization of said nucleic acid in UV imaging gel documentation device.

### BRIEF DESCRIPTION OF DRAWING FIGURES

The accompanying drawings, which are incorporated in and constitute a part of this disclosure, illustrate exemplary embodiments and, together with the description, explain the disclosed principles. The reference numbers are used throughout the figures to describe the features and components. Some embodiments of system and/or methods in accordance with embodiments of the present subject matter are now described, by way of example only, and regarding the accompanying figures, in which:

FIG. 1: illustrate/s the TEM images of Zinc bionanocomposite (ZBN) as an embodiment of the present invention.

FIG. 2: illustrate/s the time kinetics for Zinc bionanocomposite (ZBN) as fluorescent stain for E. coli as an embodiment of the present invention.

FIG. 3: illustrate/s the PXRD image distinguishing Zinc bionanocomposite (ZBN) from zinc nanoparticles (ZN)

FIG. 4: illustrates the FT-IR image distinguishing Zinc bionanocomposite (ZBN) from zinc nanoparticles (ZN)

FIG. 5: illustrates the Zeta Potential graph distinguishing Zinc bionanocomposite (ZBN) from zinc nanoparticles (ZN)

FIG. 6: illustrates the antibacterial image distinguishing Zinc bionanocomposite (ZBN) (10, 100 and 200 μg/ml) from zinc nanoparticles (ZN) (100 μg/ml)

FIG. 7: Illustrate/s the staining of (a) DNA (b) RNA (c) time kinetics for ZBN as stain for nucleic acid

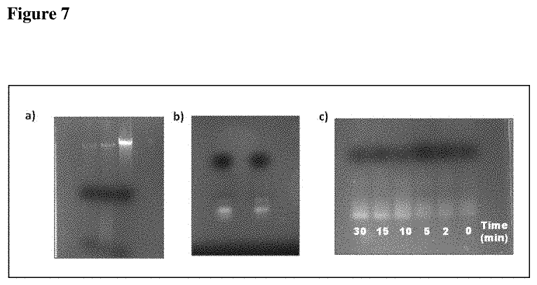

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the specific forms disclosed, but on the contrary, the disclosure is to cover all modifications, equivalents, and alternative falling within the spirit and the scope of the disclosure.

The terms “comprises”, “comprising”, “includes”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The present invention discloses a composition of zinc based fluorescent bionanocomposite and method for synthesis of said zinc bionanocomposite (ZBN). The bionanocomposite disclosed by the present invention can be used as a biologically derived nanoprobe for application (a) As ‘selective’ stain for Gram negative bacteria, the fluorescent stain is first of its kind in world (b) In an aspect of the invention, there is provided a nanoprobe, based on agriculture waste, for detecting Gram negative bacteria. In an embodiment, the nanoprobe comprises a fluorescent zinc bionanocomposite comprising a zinc salt and an extract of agriculture waste.

In an embodiment, the probe offers a rapid, sensitive and universal fluorescent detection of Gram negative bacteria “in situ” conditions. It avoids the cumbersome, time consuming procedures used conventionally that often compromises reliability and reproducibility employed during bacterial identification. A diagnostic tool is often imperative for gaining control and treating infection and the present invention inherit a one-step identification protocol for huge array of applications especially for accurately diagnosing diseases, infections applicable in health care and food safety.

In an embodiment, the fluorescent nano probe is an eco-friendly; bio-degradable fluorescent probe vis-a-vis conventional dyes used which are often reported to be toxic in nature.

In an embodiment, extract of agricultural waste comprises of straw, stems, peel and/dried leaves of agriculture waste.

In an embodiment, the bio-degradable fluorescent zinc bionanocomposite (ZBN), synthesized from agriculture waste, for selective staining of gram negative bacteria comprises:
a) At least zinc salt present in an amount ranging from 2 mmol to 80 mmol;
b) extract of agriculture waste comprising an extract of straw, stems, peel and dried leaves of said agriculture waste in a solvent, present in an amount ranging from 15 ml to 50 ml
   where ratio of said extract of agriculture waste to zinc salt comprises 0.25:1 to 5:1.

The ratio of agriculture waste to zinc salt is significant for the synthesis of the ZBN because only in this specific ratio, the desirable size range of 5-7 nm and the suitable zeta potential is achieved. This specific ratio allows the synthesis of the ZBN with negative zeta potential which ultimately retards the penetration of ZBN inside the gram negative bacterial cell.

In an embodiment, zinc salt comprises zinc nitrate, zinc chloride, zinc acetate, zinc acetylacetonate, zinc gluconate, zinc selenide preferably zinc nitrate.

In an embodiment, bionanocomposite comprises a spherical nanoparticle of said composite ranging from 5 nm to 7 nm.

In an embodiment, the plant species comprises plants from the family Rosaceae, Sapindaceae, Poaceae.

In an embodiment, the probe acts as a marker, stain, dye, biological tracker in flow cytometry, confocal microscopy and staining gram selective bacteria.

In an embodiment, the solvent comprises water, methanol, ethanol, ethyl acetate or combinations thereof.

In an embodiment, the method for the preparation of bio-degradable fluorescent zinc bio nanocomposite as described above comprises the steps of:
a) preparing a solution of zinc salt in said extract of agriculture waste in varied ratios followed by stirring to obtain a mixture, wherein the said mixture is heated at variable temperature for 45 min to 1 hour where said variable temperature comprises temperature ranging from 50° C. to 100° C.;
   wherein ratio of said extract of agriculture waste to zinc salt comprises 0.25:1 to 5:1;
b) adding the metal solution alkaline to the solution obtained in step (a) in the presence of an inert atmosphere where the formation of nanoparticle of zinc bionanocomposite is indicated by the colour change of the mixture from white to brown;
c) cooling the solution obtained in step (b) followed by filtering the washing and obtaining the nanoparticle of bionanocomposite.

In an embodiment, the metal solution alkaline comprises 2 to 30% of solution of NaOH, Na₂CO₃, NaHCO₃, hydrazine, NH₃, triethyl amine.

In an embodiment, the nanoprobe is synthesized via oxidation-reduction method wherein agriculture waste material (eco-friendly approach) was employed as a precursor for the synthesis.

In an embodiment, the variable temperature comprises a temperature ranging from 50° C. to 100° C.

In an embodiment, the reaction for formation of nano particle of bionanocomposite takes place in an inert atmosphere.

In another aspect of the present invention, there is provided a method for detecting a gram negative bacteria from biological nanoprobe as described above. The method comprises the steps of:
c) treating the overnight grown bacterial culture with the suspension of 20 micro liter of said zinc bionanocomposite (ZBN) for approximately 5-10 minutes followed by washing said culture with water;
d) detecting the bacteria through fluorescence microscope.

The zinc bionanocomposite (ZBN) may be used to fluorescently label nucleic acids (both DNA and RNA). The labeling is nontoxic and suitable for long-term tracking of cells. The material/stain/dye also can be used as nuclear stain, chromosomal stain for various applications requiring fluorescent tag of nucleic acids (eg. Cell cycle studies, Apoptosis studies etc).

In an embodiment, the invention also provides a method for the nucleic acid staining by the bio-degradable fluorescent zinc bionanocomposite ZBN. The method comprises the steps of:
a) treating approximately 500 ng of nucleic acid sample with 5 μl of diluted zinc bionanocomposite (ZBN) solution for approximately 1-30 minutes;
b) adding 1× gel loading dye to the solution obtained from step (a) followed by running on agarose gel electrophoresis and visualization of said nucleic acid in UV imaging gel documentation device.

In an embodiment, the nucleic acid comprises DNA, RNA.

### Applications

The synthesized zincbionanocomposite (ZBN) can be used as Marker/Stain/Dye for electron microscopy and has ability to replace commonly used toxic metals like osmium.
Marker/Stain/Dye/biological tracker for flow-cytometry
Marker/Stain/Dye/biological tracker for confocal microscopy.
Nanoprobe/stain/dye/biological tracker for staining gram negative bacteria.
Probe/ /biological tracker for live cell imaging.
Universal stain for nucleic acids (DNA/RNA/Oligonucleotides), nuclear staining, chromosomal staining.
Replacement of radioisotopes for labeling nucleotides for various applications s.a. sequencing and in real time quantification of nucleic acid during qPCR, droplet digital PCR, HRM, LAMP, tHDA etc.
Endotoxin detection marker/probe.
Immuno-staining purpose.

The invention is further explained by the following non-limiting examples:

### Example 1

The experiment was performed to compare the fluorescent stain with the commercially available stains. It was found that most of the conventional stains suffer/s from some critical limitations. The overview of the drawbacks for individual fluorescent stains is enlisted in the table 1 below:

TABLE 1
Comparison of ZBN with other fluorescent bacterial stains

| | | | | Target bacteria | | Drawback(s) | | | |
|---|---|---|---|---|---|---|---|---|---|
| Fluorescent | Type of | Target vitality | | Gram | Gram | Staining | | Light | |
| probe | probe | Live | Dead | positive | negative | time | Toxicity | sensitivity | Ref |
| DAPI | Synthetic | [symbol] | [symbol] | [symbol] | [symbol] | 15-30 | Potential | [symbol] | 1-2 |
| | | | | | | min | mutagen | | |
| SYTO 9 | Synthetic | [symbol] | [symbol] | [symbol] | [symbol] | 1-30 | Potential | [symbol] | 3-4 |
| | | | | | | min | mutagen | | |
| PI | Synthetic | [symbol] | [symbol] | [symbol] | [symbol] | 5 min | Potential | [symbol] | 5-6 |
| | | | | | | | mutagen | | |
| Rhodamine | Synthetic | [symbol] | [symbol] | [symbol] | [symbol] | 30 min | Toxic | [symbol] | 7-8 |
| 123 | | | | | | | | | |
| DMAO | synthetic | [symbol] | [symbol] | [symbol] | [symbol] | 15-30 | Not | [symbol] | 9-10 |
| | | | | | | min | known | | |
| Acridine | Synthetic | [symbol] | [symbol] | [symbol] | [symbol] | 2 min | Toxic | [symbol] | 11-13 |
| Orange | | | | | | | | | |
| Hoechst | Synthetic | [symbol] | [symbol] | [symbol] | [symbol] | 10-30 | Potential | [symbol] | 14 |
| | | | | | | min | mutagen | | |
| Brazilein | Natural | [symbol] | [symbol] | [symbol] | ✓ | 15 min | [symbol] | ✓ | 15 |
| ZBN | Natural | ✓ | ✓ | [symbol] | ✓ | >10 | [symbol] | [symbol] | Present |
| | | | | | | min | | | invention |

ZBN stain is synthesized from agriculture waste which is a greener method and hence has shown to be non-toxic. Most importantly, the ZBN stain in the present invention is selective for Gram negative bacteria and there are no stains reported so far for selectively staining gram negative stain. The usage is very simple single step protocol.

Further, no dark conditions are required which is conventionally a necessary requirement for all current available fluorescent stains. The synthesized ZBN stain is very rapid and detects the Gram negative bacteria within just <10 minutes. Since there are almost no selective gram negative stains available in the market, it has huge commercial value for bacterial diagnostics applicable in agriculture, food safety, water purification and healthcare.

### Example 2

### Synthesis of Zinc Bionanocomposite (ZBN)

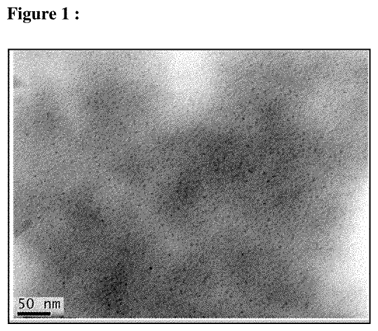

Optimal reaction conditions for the formation of ZBN were determined by conducting UV-Visible and TEM studies. ZBN synthesis was done under variable range of reaction conditions i.e. different ratios of zinc salts and extracts from agriculture waste at different temperature for variable time. ZBN was optimised when 5 ml of 2 mmol/L solution of Zinc salt (examples of zinc salt that may be applied and are not limited to zinc nitrate/zinc chloride/zinc acetate/zinc sulphate/zinc carbonate/zinc citrate/zinc acetylacetonate/zinc chlorate/zinc gluconate/zinc oleate/zinc phosphate) was used with extract of plant from the family (Rosaceae/Sapindaceae/Poaceae)) based agricultural waste (can consists of straws and/or stems and/or peels and/or dried leaves). The mixture obtained was stirred on magnetic stirrers and heated at variable temperature (room temperature, 50° C., 60° C. and 80° C. and 100° C.) for 1 h. The mixture was made alkaline with 2-30% of solution of (NaOH/Na₂CO₃/NaHCO₃hydrazine/NH₃/triethyl amine). The reaction was conducted in an inert atmosphere. Formation of the particles was observed as a colour change of the reaction mixture from white to brownish. After the reaction was over, the reaction was cooled, centrifuged and washed with water and dried. The TEM image of ZBN (FIG. 1) shows spherical particles of size ranging from 5-7 nm.

ZBN was characterized via sophisticated techniques.

The plant based agricultural waste not only acts as a reducing agent but also as a stabilizing ligand to form a zinc metal based coordinate complex for bionanocomposite formation.

### Example 3

### Synthesis of Zinc Oxide Nanoparticles (ZN) (Negative Control)

ZN was synthesised by the co-precipitation method (16) wherein ZN was synthesised when 5 ml of 2 mmol/L solution of zinc salt that may be applied and are not limited to (zinc nitrate/zinc chloride/zinc acetate/zinc sulphate/zinc carbonate/zinc citrate/zinc acetylacetonate/zinc chlorate/zinc gluconate/zinc oleate/zinc phosphate) was constantly stirred with NaOH aqueous solution that was added drop wise. The white precipitate i.e ZN appeared and was washed with deionised water. The ZN was filtered and dried thoroughly. ZN was characterised via sophisticated techniques.

### Example 4

### Characterization of Materials ZBN and ZN

The materials were Characterised via:

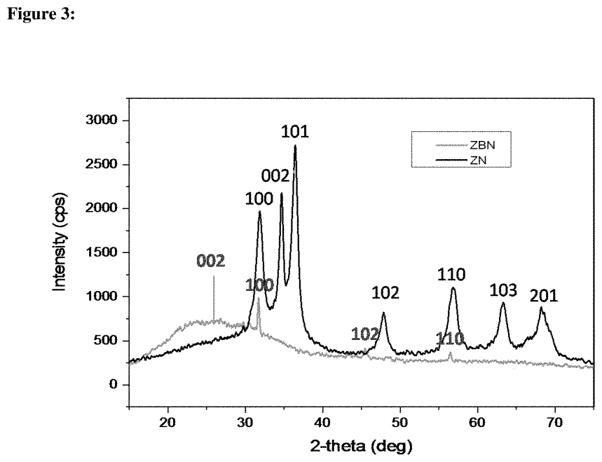

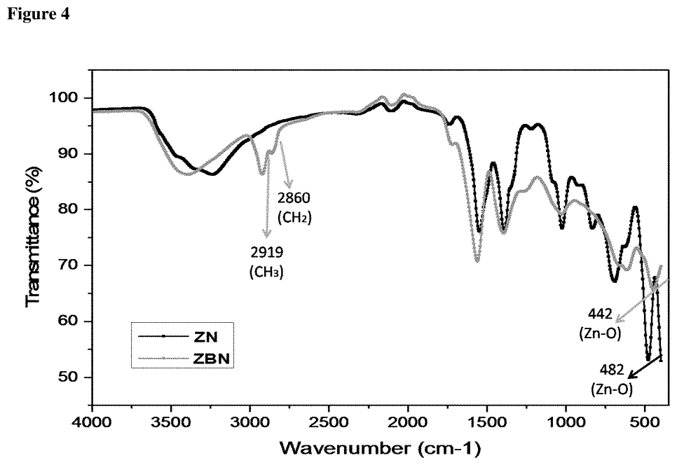

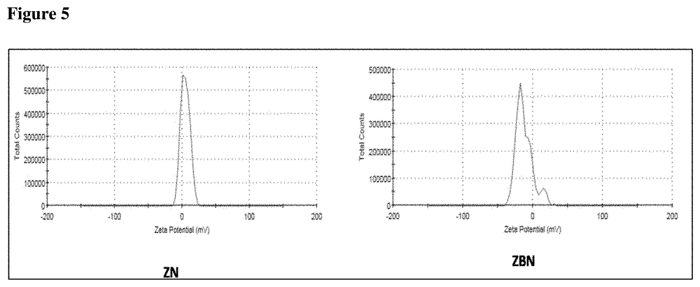

(a) Powder Xray Diffraction (PXRD), showed in FIG. 3 clearly distinguishes ZBN from ZN. The PXRD of ZBN does not show the (101), (103) and (201) peaks which are otherwise present in ZN. Moreover the hump at 2 theta 25.779 i.e (002) which has been shifted in ZBN clearly indicates the presence of carbon in ZBN.
(b) Fourier Transform Infrared Spectroscopy (FT-IR) showed in FIG. 4 for ZBN indicates clearly that peaks at wavelength 2919 cm⁻¹ and 2860 cm⁻¹ depicts the presence of alkyl groups which are otherwise missing in ZN. Moreover there is a shift in Zn—O stretching peaks from 482 cm⁻¹ to 442 cm⁻¹ clearly indicating that ZBN is different from ZN
(c) Zeta Potential measurement showed in FIG. 5 indicates the major difference between ZBN and ZN. Since the ZBN has shown the negative surface potential of (−12.3 mV) while the ZN has shown the positive surface potential of (+4.70 mV). This negative zeta potential did not allow ZBN to penetrate the bacterial cell and due to which it was not antibacterial.

### Example 5

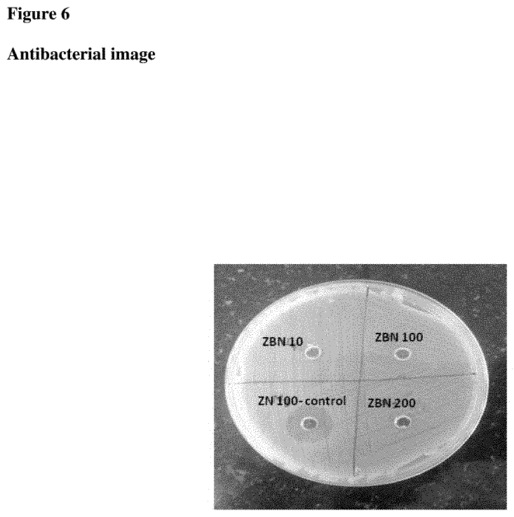

Antibacterial Activity of ZBN and ZN was determined against pathogenic bacterium, Escherichia coli tested for antimicrobial activity by Kirby Bauer well-diffusion method. The overnight grown culture of the test bacterium were adjusted to an optical density of 0.1 at 600 nm (˜1×10⁸ CFU/ml) and swabbed uniformly onto Mueller-Hinton agar plates. Wells of 6-mm diameter were punctured into the agar plates and 100 μg/ml of ZN and ZBN nanoparticles (different concentrations 50, 100 and 200 μg/ml) were added to the wells and the plates were incubated at 37° C. for 24 h. The clear zone of bacterial inhibition was measured for analysis. As depicted in FIG. 6, a clear zone of bacterial inhibition was obtained for ZN nanoparticles (indicating antibacterial activity) however, no zone of antibacterial activity was observed for the ZBN nanoparticles. This indicates that ZBN nanoparticles do not have any antibacterial activity.

### Example 6

Time Kinetic for ZBN as Fluorescent Stain for E. coli

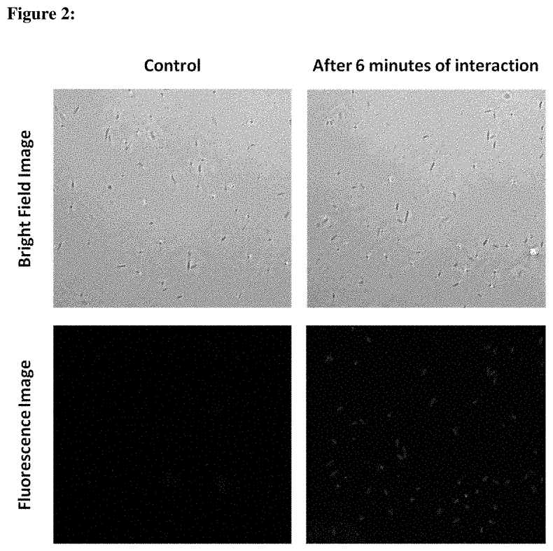

Kinetic effect of ZBN for fluorescent staining of E. coli was done by employing optimized concentration of ZBN herein the nanocomposite was allowed to interact with E. coli for 1 to 10 minutes. Overnight grown bacterial culture was casted on glass slide and was dried. To this 20 μL of ZBN suspension was added and allowed to interact for variable time points (1-10 minutes). Slides were viewed under the fluorescence microscope. The Gram negative bacteria (here E. coli) were best observed at 6 minute (as shown in FIG. 2).

### Example 7

In this aspect of the present invention, the zinc bionanocomposite (ZBN) provided a natural, bio-degradable, no wash stain for nucleic acids which can be used to trace, track and quantify nucleic acids within short time duration. Further, the material may be used as a fluorescent nanoprobe for various applications like nuclear imaging, chromosomal/extra-chromosomal staining, apoptosis studies, label-free tagging of nucleic acid probes etc. The present ZBN biostain can potentially replace toxic chemical dyes like ethidium bromide (refer table 2 for comparison with other fluorescent nucleic acid stains).

TABLE 2
Comparison of ZBN with other fluorescent nucleic acid stainsas disclosed in the reference
documents, details of which is mentioned below.

| | | Target | Drawback | | | |
|---|---|---|---|---|---|---|
| | Type of | Nucleic | Staining | | Light | |
| | probe | acid | time | Toxicity | Sensitivity | Ref |
| Hoechst | Synthetic | DNA | 5-15 min | Potentially | ✓ | 14 & 17-19 |
| | | | | mutagenic | | |
| | | | | and | | |
| | | | | carcinogenic | | |
| DAPI | Synthetic | DNA | 5-15 min | Potentially | ✓ | 1-2 &20-21 |
| | | | | mutagenic | | |
| NucSpot ® | Synthetic | DNA | 10 | Toxic | ✓ | 22 |
| Live Cell | | | minutes | | | |
| Nuclear | | | or | | | |
| Stains | | | longer | | | |
| RedDot ™1 | Synthetic | DNA | 5-30 min | Toxic | ✓ | 23 |
| Far-Red | | | | | | |
| Nuclear Stain | | | | | | |
| DMAO | Synthetic | DNA | 30 min | Unknown | ✓ | 24 |
| BactoView ™ | Synthetic | DNA | 30 min | Toxic | ✓ | 25 |
| Live Green | | | | | | |
| BactoView ™ | Synthetic | DNA | 30 min | Toxic | ✓ | 26 |
| Live Red | | | | | | |
| EtBr | Synthetic | DNA/RNA | 30 min | Potent | ✓ | 27 |
| | | | | mutagen | | |
| SyBr green I | Synthetic | DNA | 1-40 min | Potent | ✓ | 28-29 |
| | | | | mutagen | | |
| SyBr Green | Synthetic | RNA | 1-40 min | Unknown | ✓ | 30 |
| II stain | | | | | | |
| SyBr ™ Gold | Synthetic | DNA/RNA | 1-40 min | Unknown | ✓ | 31 |
| SyBr Safe | Synthetic | DNA | 1-40 min | Potent | | 32 |
| DNA Gel | | | | mutagen | | |
| Stain | | | | | | |
| Thiazole | Synthetic | DNA/RNA | 30 min-1 h | Unknown | ✓ | 33-34 |
| Orange | | | | | | |
| ZBN | Natural | DNA/RNA | 30 min | [symbol] | [symbol] | Present |
| | | | | | | Invention |

### FULL REFERENCES OF THE DOCUMENTS REFERRED IN THE ABOVE COMPARISON TABLE 1 AND 2

(1) Biotium Protocol: Staining Cells with Hoechst or Dapi Nuclear Stains. https://biotium.com/tech-tips/protocol-staining-cells-with-hoechst-or-dapi-nuclear-stains/.
(2) Dojindo Molecular Technologies Cell Staining. https://www.dojindo.com/Protocol/CellStaining Protocol.pdf.
(3) Stiefel, P.; Schmidt-Emrich, S.; Maniura-Weber, K.; Ren, Q. Critical Aspects of Using Bacterial Cell Viability Assays with the Fluorophores Syto9 and Propidium Iodide. BMC microbiology 2015, 15 (1), 36.
(4) Thermo Fisher Scientific Syto™ 9 Green Fluorescent Nucleic Acid Stain: Manuals and Protocols. https://www.thermofisher.com/order/catalog/product/S34854#/S34854.
(5) Thermo Fisher Scientific Propidium Iodide Nucleic Acid Stain. https://assets.thermofisher.com/TFS-Assets/LSG/manuals/mp01304.pdf.
(6) Sigma Aldrich Product Information: 79214 Bacteria Stain Propidium Iodide Solution https://www.sigmaaldrich.com/content/dam/sigma-aldrich/docs/Sigma/Datasheet/10/79214dat.pdf.
(7) Lampidis, T. J.; Bernal, S. D.; Summerhayes, I. C.; Chen, L. B. Selective Toxicity of Rhodamine 123 in Carcinoma Cells in Vitro. Cancer research 1983, 43 (2), 716-720.
(8) Matsuyama, T. Staining of Living Bacteria with Rhodamine 123. FEMS microbiology letters 1984, 21 (2), 153-157.
(9) PromoCell Bacteria Live/Dead Staining Kit: Instruction Manual. https://www.promocell.comproduct/bacteria-live-dead-staining kit/.
(10) Biotium Dmao, 2 Mm in Dmso: Product Information https://biotium.com/product/dmao-2 mm-in-dmso/.
(11) Lauer, B.; Reller, L.; Mirrett, S. Comparison of Acridine Orange and Gram Stains for Detection of Microorganisms in Cerebrospinal Fluid and Other Clinical Specimens. Journal of clinical microbiology 1981, 14 (2), 201-205.
(12) IHC World Life Science Products and Services Abc of Safety in the Biological Sciences. http://www.ihcworld.com/royellis/ABCSafe/chemicals/acridine-orane.htm (accessed 30/1/2020).
(13) Thermo Fisher Scientific Thermo Scientific™ Remel Acridine Orange:Manuals and Protocols https://www.fishersci.comlsho/roducts/thermo-scientific-remel-acridine-orange-250ml-bottle-ea/r40010.
(14) Thermo Fisher Scientific Invitrogen™ Molecular Probes™ Hoechst 33342, Trihydrochloride, Trihydrate: Manuals and Protocols. https://www.fishersci.ca/shop/products/molecular-probes-hoechst33342-trihydrochloride-trihydrate-2/h3570.
(15) Jahangir Ali, F. B.; Rabidass, A. D. P.; Savithri, H. S. Plant Based Dye for Staining of Biological Samples, Extraction Method and Use Thereof. 2016.
(16) A. Jafar Ahamed and P. Vijaya Kumar, Synthesis and characterization of ZnO nanoparticles by co-precipitation method at room temperature, Journal of Chemical and Pharmaceutical Research, 2016, 8(5):624-628.
17. https://biotium.com/product/hoechst/
18. https://assets.thermofisher.com/TFS-Assets/LSG/manuals/MAN0011717 Hoechst 33342 UG.pdf
19. https://pubmed.ncbi.nlm.nih.gov/4054243/
20. https://biotium.com/product/dapi/
21. https://pubmed.ncbi.nlm.nih.gov/4054243/
22. https://biotium.com/product/nucspot-live-cell-nuclear-stains/
23. https://biotium.com/product/reddottml-far-red-nuclear-stain-200x-in-h2o/
24. https://biotium.com/product/dmao-2 mm-in-dmso/
25. https://biotium.com/product/bactoview-live/
26. https://biotium.com/product/bactoview-live/
27. https://www.sigmaaldrich.com/technical-documents/articles/biologv/ethidium-bromide.html
28. https://www.thermofisher.com/document-connect/document-connect.html?url=https%3A%2F%2Fassets.thermofisher.com%2FTFS-Assets%2FLSG%2Fmanuals%2Fmp07567.pdf&title=U1lCUiBHcmVlbiBJIE51Y2xlaWMgQWNpZCBHZWwgU3RhaW4=
29. https://www.sigmaaldrich.com/technical-documents/protocols/biologv/svbr-green-qpcr.html?gclid=CjwKCAiAp4KCBhB6EiwAxRxbpLiVtLM2a40p3NwGObD77AMXO71EAk1IppwkOO7Kbs6poXkx5sozrhoC-IcQAvD_BwE
30. https://www.thermofisher.com/document-connect/document-connect.html?url=https%3A%2F%2Fassets.thermofisher.com%2FTFS-Assets%2FLSG%2Fmanuals%2Fmp07568.pdf&title=U1lCUiBHcmVlbiBJSSBSTkEgR2VsIFNOYWlu
31. https://www.thermofisher.com/document-connect/document-connect.html?url=https%3A%2F%2Fassets.thermofisher.com%2FTFS-Assets%2FLSG%2Fmanuals%2Fmp11494.pdf&title=U1lCUiBHb2xkIE51Y2xlaWMgQWNpZCBHZWwgU3RhaW4=
32. https://www.thermofisher.com/nz/en/home/life-science/dna-rna-purification-analysis/nucleic-acid-gel-electrophoresis/dna-stains/sybr-safe.html
33. https://onlinelibrary.wiley.com/doi/epdf/10.1002/cyto.990070603
   High-sensitivity two-color detection of double-stranded DNA with a confocal fluorescence gel scanner using ethidium homodimer and thiazole orange.
34. https://biotium.com/product/thiazole-orange-10-mm-in-dmso/

### Example 8

### Nucleic Acid Staining

The zinc bionanocomposite (ZBN) disclosed by the present invention can be used as a biologically derived, biodegradable, no wash, stable universal nucleic acid stain for both DNA and RNA. The staining method comprises of the following steps:

a) 500 ng of nucleic acid sample was mixed with 5 μl of diluted ZBN solution and allowed to interact for variable time points (30, 15, 10, 5, 2 and 0 min);
b) After incubation, the sample mixed with 1× gel loading dye and run on agarose gel electrophoresis followed by visualization of DNA/RNA in UV imaging gel documentation device.

1. A bio-degradable fluorescent zinc bionanocomposite, synthesized from agriculture waste, for selective staining of gram negative bacteria comprising:
a) At least zinc salt present in an amount ranging from 2 mmol to 80 mmol;
b) extract of agriculture waste comprising an extract of straw, stems, peel and dried leaves of said agriculture waste in a solvent, present in an amount ranging from 15 ml to 50 ml;
where ratio of said extract of agriculture waste to zinc salt comprises 0.25:1 to 5:1.

2. The zinc bionanocomposite as claimed in claim 1, wherein said zinc salt comprises zinc nitrate, zinc chloride, zinc acetate, zinc sulphate, zinc carbonate, zinc citrate, zinc acetylacetonate, zinc chlorate, zinc gluconate, zinc oleate, zinc phosphate.

3. The zinc bionanocomposite as claimed in claim 1, wherein said plant species comprises plants from the family Rosaceae, Sapindaceae, Poaceae.

4. The zinc bionanocomposite as claimed in claim 1, wherein said bionanocomposite comprises a spherical nanoparticles of size ranging from 5 nm to 7 nm.

5. The zinc bionanocomposite as claimed in claim 1, wherein solvent comprises water, methanol, ethanol, ethyl acetate or combinations thereof.

6. A method for the preparation of bio-degradable fluorescent zinc bionanocomposite as claimed in claim 1, comprising the steps of:
a) preparing a solution of zinc salt in said extract of agriculture waste in varied ratios followed by stirring to obtain a mixture, wherein the said mixture is heated at variable temperature for 45 min to 1 hour where said variable temperature comprises temperature ranging from 50° C. to 100° C.;
where ratio of said extract of agriculture waste to zinc salt comprises 0.25:1 to 5:1;
b) adding the metal solution alkaline to the solution obtained in step (a) in the presence of an inert atmosphere where the formation of nanoparticle of bionanocomposite is indicated by the colour change of the mixture from white to brown;
c) cooling the solution obtained in step (b) followed by filtering the washing and obtaining the nanoparticle of bionanocomposite.

7. The method as claimed in claim 6, wherein said metal solution alkaline comprises 2 to 30% of solution of NaOH, Na₂CO₃, NaHCO₃, hydrazine, NH₃, triethyl amine.

8. A method for the detection of gram negative bacteria by the bio-degradable fluorescent zinc bionanocomposite as claimed in claim 1, comprising:
a) treating the overnight grown bacterial culture with the suspension of 20 micro liter of said zinc bionanocomposite for approximately 5-10 minutes followed by washing said culture with water;
b) detecting the bacteria through fluorescence microscope.

9. A method for the nucleic acid staining by the bio-degradable fluorescent zinc bionanocomposite as claimed in claim 1, comprising the steps of:
a) treating approximately 500 ng of nucleic acid sample with 5 μl of diluted zinc bionanocomposite solution for approximately 1-30 minutes;
b) adding 1× gel loading dye to the solution obtained from step (a) followed by running on agarose gel electrophoresis and visualization of said nucleic acid in UV imaging gel documentation device.

10. The method as claimed in claim 8, wherein said nucleic acid comprises DNA, RNA.
